(19) United States
(12) Patent Application Publication
(10) Pub. No.: US 2022/0129068 A1
(43) Pub. Date: Apr. 28, 2022

(54) EYE TRACKING FOR DISPLAYS

(71) Applicant: Hewlett-Packard Development Company, L.P., Spring, TX (US)

(72) Inventors: Jonathan Michael Anderson, Fort Collins, CO (US); Louis M. Gaiot, Fort Collins, CO (US); Cheng Huang, Palo Alto, CA (US)

(73) Assignee: Hewlett-Packard Development Company, L.P., Spring, TX (US)

(21) Appl. No.: 17/416,689

(22) PCT Filed: Jul. 11, 2019
(86) PCT No.: PCT/US2019/041289

§ 371 Date: Jun. 21, 2021

(51) Int. Cl. G06F 3/01; G02B 27/00; G02B 27/01; A61B 3/11; A61B 3/113; G06F 3/04817; G06F 3/0482

(52) U.S. Cl. CPC G06F 3/013; G02B 27/0093; G02B 27/0179; G02B 27/0101; G02B 27/017; G02B 2027/0138; A61B 3/113; G06F 3/04817; G06F 3/0482; G02B 2027/0187; A61B 3/112

(57) ABSTRACT

In example implementations, a display is provided. The display includes a camera, a communication interface, and a processor. The camera is to capture a first image of a head-mounted device (HMD) wearable by a user. The communication interface is to receive pupil data from the HMD. The processor is communicatively coupled to the camera and the wireless communication interface. The processor is to determine abound of a field-of-view based on the first image of the HMD, track an eye of the user based on the field-of-view and the pupil data to determine a location of focus of the user, and move a second image to the location of focus on the display.

### BACKGROUND

Displays are used to present information, graphics, video, and the like. For example, graphical user interfaces (GUIs) can be presented on a display, and the user may interact with the GUIs to execute applications. The size of displays has also grown over the years. For example, displays have grown from 19 inches to well over 30 inches. In addition, displays have changed from a 4:3 aspect ratio to larger wide screen and ultra-wide screen aspect ratios.

### BRIEF DESCRIPTION OF THE DRAWINGS

FIG. 1 is a block diagram of an example system to adjust an image on the display based on tracking the eye of a user of the present disclosure;

FIG. 2 is a block diagram of a display of the present disclosure;

FIG. 3 illustrates an example of controlling an image on the display based on tracking the eye of the user of the present disclosure;

FIG. 4 illustrates an example of moving an image on the display based on tracking the eye of the user of the present disclosure;

FIG. 5 is a flow chart of an example method for moving an image on a display based on tracking the eye of a user; and

FIG. 6 is a block diagram of an example non-transitory computer readable storage medium storing instructions executed by a processor to move a graphical image on a display based on tracking the eye of a user.

### DETAILED DESCRIPTION

Examples described herein provide an apparatus and method to adjust an image based on tracking the eye of a user. As noted above, displays can be used to present information. Over the years, the size of displays has grown larger and larger. Thus, a user may tend to focus on certain portions of the display when viewing a very large display.

Examples herein provide a display with a camera that works with a head-mounted device (HMD) to track the eyes of a user. The camera can provide overall context or a field-of-view of the user. The HMD may provide information to the display related to where the pupils of the eyes of the user are focused. Based on the overall field-of-view and the pupils of the eyes, the eyes of the user may be tracked relative to the images on the display.

Based on the eye-tracking data, the display may adjust an image (e.g., a graphical image, an icon, and the like). For example, the image may be a cursor that is controlled by the user's eyes. In another example, the image may be an icon or menu of icons in a graphical user interface. For example, if the user tends to look more on the right side of the display, the icons can be automatically moved to the right side of the display so the user can easily find the desired icons.

In another example, the eye-tracking process may provide commercial benefits. Eye-tracking, as discussed in further details below, includes a set of operations or results of those operations that may indicate a position, orientation, or attributes of the eye. For example, the eye-tracking process may be used to collect eye-tracking data.

Companies may offer to pay the user for the eye-tracking data. Based on the eye-tracking data, the companies may know where the user tends to look on the display or a web browser. The company may then offer to sell advertising space on portions of the web browser that are viewed most often by a user. Each user may have a unique eye-tracking profile that indicates which portions of the web browser or GUI are viewed most often. The advertisements may then be moved to those locations for a particular user based on the user's unique eye-tracking profile.

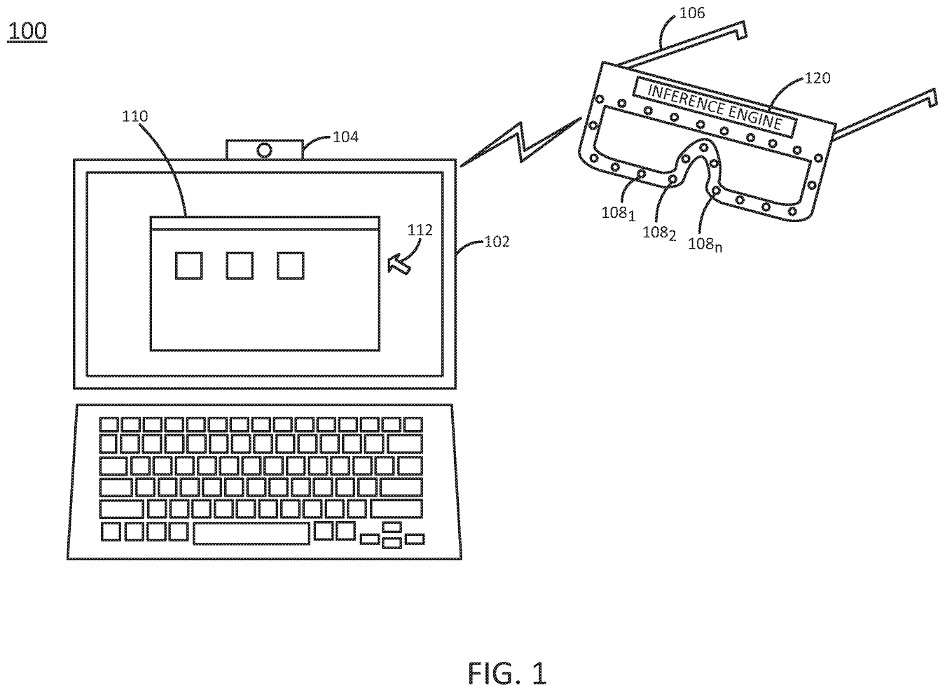

FIG. 1 illustrates an example system 100 of the present disclosure. In an example, the system 100 may include a display 102 and an HMD 106. The display 102 may be a monitor that can be used to display images 110 and 112. The images 110 and 12 may be graphics, images, videos, text, graphical user interfaces (GUIs), digital advertisements, and the like. The display 102 may work with a computing device or be part of an all-in-one computing device.

In an example, the display 102 may include a camera 104. The camera 104 may be an external camera or may be built in as part of the display 102. In an example, the camera 104 may be mounted towards a top center of the display 102. The camera 104 may capture images of the HMD 106. The images may be analyzed to determine an orientation of the HMD 106, which can then be used to determine a field-of-view of the user, as discussed in further details below.

In an example, the HMD 106 may be wearable by a user. For example, the HMD 106 may be implemented as glasses with or without lenses. The user may wear the HMD 106 while viewing the display 102. The HMD 106 may include sensors 108₁ to 108ₙ (hereinafter individually referred to as a “sensor 108” or collectively referred to as “sensors 108”). Although a plurality of sensors 108 are illustrated in FIG. 1, it should be noted that the HMD 106 may include a single sensor.

In an example, the sensors 108 may be the same type of sensor or may be different types of sensors. The sensors 108 may collect pupil data as well as other types of biometric data of the user. For example, the sensors 108 may include an eye-tracking sensor, such as a camera that captures images of the eyes or pupils of a user or a near infrared light that can be directed towards the pupils to create a reflection that can be tracked by an infrared camera. The eye-tracking sensor may track the movement of the eye or eyes of a user. The movement of the eyes can be converted into a gaze vector that indicates where the user is looking. The gaze vector may then be wirelessly transmitted to the display 102.

As noted above, the field-of-view of the user can be determined by analyzing images of the HMD 106. Also, the display 102 may know what is being shown on the display 102. With the gaze vector and the field-of-view of the user to provide context, the display 102 may calculate a location of focus, or focus location, on the display 102. In other words, the location of focus may be a location that the HMD 106 is intended to look at based on the calculated gaze vectors and field-of-view of the user.

In an example, the location of focus may then correspond to a location on the display 102. In other words, the display 102 may correlate the location of focus intended by HMD 106 to an actual location on the display 102 (e.g., an x-y coordinate, a pixel location and the like). Thus, hereinafter the terms “location of focus” and “focus location” may be interchangeably used to also indicate the corresponding location on the display 102. The location of focus may be applied in a variety of different ways, as discussed in further details below.

In an example, the sensors 108 may include other types of sensors to collect biometric data. For example, the sensors 108 may include a pupillometry sensor. The pupillometry sensor may measure pupil dilation.

In an example, the sensors 108 may include heart rate monitors, blood pressure monitors, electromyography (EMG) sensors, and the like. The sensors 108 may be used to measure biometric data such as heart rate, blood pressure, muscle activity around the eyes, and the like. The biometric data may be analyzed by an inference engine 120 that is trained to determine a cognitive load of the user. The inference engine 120 may be trained with training data of biometric data and cognitive loads, such that inference engine 120 may determine the cognitive load based on the biometric data.

In an example, the inference engine 120 may be stored in the HMD 106. The biometric data and pupil data may be analyzed locally by the inference engine 120 in the HMD 106. The cognitive load can be determined locally by the inference engine 120 in the HMD 106. Then the cognitive load can be transmitted by the HMD 106 to the display 102 via a wireless communication path between the HMD 106 and the display 102. In another example, the inference engine 120 may be stored in the display 102. The biometric data and pupil data can be transmitted to the display 102 and the inference engine 120 in the display 102 may calculate the cognitive load of the user.

In an example, the display 102 may make adjustments or changes to an image located at a location on the display 102 that corresponds to the focus location of the user based on the cognitive load. For example, display 102 may make the image more interesting if the cognitive load is too low or may the image less interesting if the cognitive load is too high.

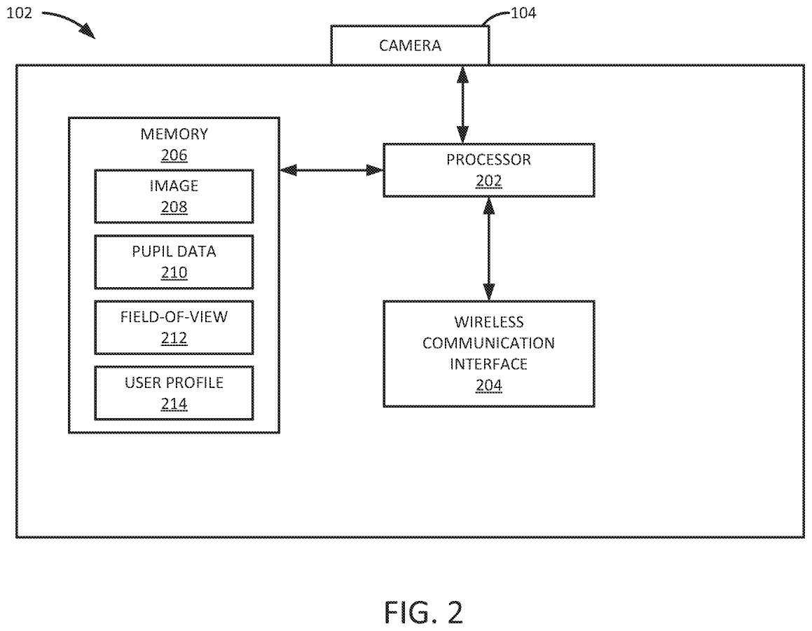

FIG. 2 illustrates a block diagram of the display 102. In an example, the display 102 may include the camera 104, as illustrated in FIG. 1. The display 102 may also include a processor 202, a wireless communication interface 204, and a memory 206. The processor 202 may be part of the display 102 in devices such as an all-in-one computer. In another example, the processor 202 may be part of a computing device that is communicatively coupled to the display 102. In another example, the processor 202 may be part of the display 102 and may operate independent of any computing device.

The processor 202 may be communicatively coupled to the wireless communication interface 204 and to the memory 206. In an example, the wireless communication interface 204 may be any type of wireless transceiver that may transmit and receive data over a wireless communication path. For example, the wireless communication interface 204 may be a WiFi radio, a Bluetooth radio, and the like.

In an example, the memory 206 may be a non-transitory computer readable medium. For example, the memory 206 may be hard disk drive, a solid state drive, a read-only memory (ROM), a random access memory (RAM), and the like.

The memory 206 may include an image 208, pupil data 210, a field-of-view 212, and a user profile 214. The image 208 may be an image of the HMD 106 that is captured by the camera 104. The image 208 may be analyzed to determine an orientation (e.g., if the HMD 106 is pointing left, right, up, down, or any combination thereof) of the HMD 106. The image 208 may also be analyzed to determine an estimated distance of the HMD 106 from the camera 104 based on the size of the HMD 106 in the image 208 and a known size of the HMD 106. Based on the orientation of the HMD 106 and a distance of the HMD 106 from the camera 104, the processor 202 may calculate a bound of the field-of-view of the user. The bound of the field-of-view and the field-of-view may be stored in the field-of-view 212.

In an example, the pupil data 210 may include the gaze vector that is received from the HMD 106. In an example, the pupil data 210 may include other pupil data such as the pupillometry data, described above. As noted above, with the gaze vector and the bound of the field-of-view, the processor 202 may determine a location of focus on the display 102 of the user.

In an example, the image 208, the pupil data 210 and the field-of-view 212 may be continuously tracked and updated. For example, the image 208 may be updated as the camera 104 periodically (e.g., every 2 seconds, every 10 seconds, every 30 seconds, and the like) captures images of the HMD 106.

In an example, the location of focus of the user may be tracked over time. The tracked locations of focus may then be stored as part of the user profile 214. For example, the user profile 214 may be an eye-tracking profile that provides data related to a favored location of focus of the user. The favored location of focus may be a location on the display that the user focuses on for a greater amount of time than a threshold amount of time.

For example, the display 102 may be divided into a plurality of quadrants. The number of times that the location of focus is in a specific quadrant can be tracked. The quadrant that has the location of focus more than 50% of the time can be considered a favored location of focus. In an example, the quadrant that is the location of focus the most number of times (overall aggregate or during a specified time period) can be the favored location of focus.

In an example, the user profile 214 may include favored location of focus for a particular image 110. For example, the image 110 may be an application window or a web browser. The image 110 can be divided into quadrants and the favored location of focus within the image 110 can be determined, as described above.

In an example, the user profile 214 can be used to rearrange images 110 and 112 in the display 102. For example, if the favored location of focus on the display 102 is the top center of the display 102, the processor 202 may move the images 110 and 112 to the top center of the display 102.

In another example, the user profile 214 can be transmitted to a third party or can be sold to the third party. For example, the third party may be an advertisement company or a search engine that sells ads on a web browser. In exchange for money, the user may sell the information stored in the user profile 214.

For example, the favored location of focus of the user in a web browser may be the bottom center of the web browser. The user may tend to read ahead to the bottom of a web page. Based on the favored location of focus, an advertisement may be placed in the bottom center of the web page where the user tends to look most often in the web browser.

It should be noted that the display 102 has been simplified for ease of explanation and that the display 102 may include more components that are not shown. For example, the display 102 may include light emitting diodes, additional display panels, a power supply, and so forth.

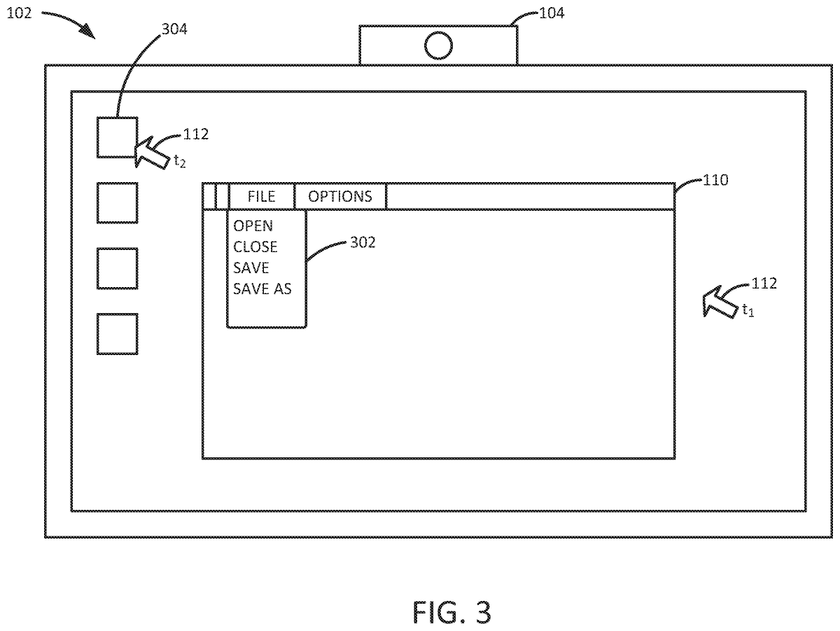

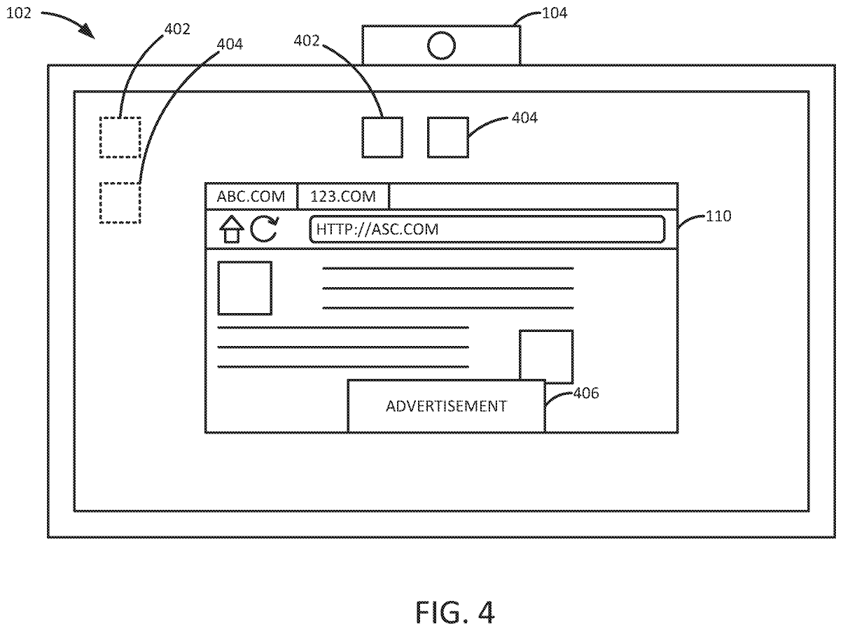

FIGS. 3 and 4 illustrate examples of how the location of focus of the user can be used to move images 110 and 112, as described above. FIG. 3 illustrates an example, where the image 112 is a cursor that is overlaid on other images shown on the display 102. In an example, a graphical user interface shown on the display 102 may provide an option to enable cursor control via eye-tracking.

In an example, the location of focus may be detected to be on the image 112 (also referred to herein as the cursor 112) at time 1 (t₁). For example, the processor 202 may receive gaze vector data from the HMD 106 and determine the bound of a field-of-view of the user based on images of the HMD 106 captured by the camera 104. The processor 202 may determine based on the gaze vector data and the field-of-view that the location of focus is on the display where the cursor 112 is located at time tₜ. The display 102 may know what images are shown on the display and compare the known displayed images to the location of focus. Based on the comparison, the display 102 can determine that the cursor 112 is being shown at the location of focus on the display 102. With the cursor control via eye-tracking enabled, the display 102 may determine that the user is looking at the cursor 112 to move the cursor 112.

The display 102 may continuously perform eye-tracking by capturing images of the HMD 106 for field-of-view and receiving gaze vector data from the HMD 106. The display may move the cursor 112 on the display 102 as the eye-tracking detects that the user is looking to a different location on the display 102. For example, the user may be moving the cursor 112 to select an icon 304 as shown in FIG. 3. Thus, at time t₃, the cursor 112 may be moved to be overlaid on the icon 304.

In an example, the user may release control of the cursor 112 by closing his or her eyes for greater than a predetermined amount of time (e.g., 3 seconds) or by turning their head away from the display 102 such that the field-of-view does not include the display 102. Releasing control of the cursor 112 may prevent the cursor 112 from moving around the display 102 as the user is working in another window or using another application shown on the display 102.

In an example, the eye-tracking may also be used to display a menu 302. For example, the image 110 may be a window or a graphical user interface (also referred to as GUI 110). When the location of focus is determined to be on the GUI 110, the display 102 may open the menu 302.

In one example, the cursor 112 may be moved and overlaid on a menu option in the image 110. In one example, when the focus location or gaze vector is determined to be on the cursor 112 that is located over a menu option of the image 110 fora predetermined amount of time (e.g., greater than 3 second), then the an action may be performed. For example, the menu 302 may be opened.

In another example, the location of focus may be on the icon 304. The display 102 may display a menu associated with the icon 304. For example, the menu may provide options to open the folder, start the application, and the like. The user may select the “enter” key on the keyboard to select the option.

FIG. 4 illustrates examples of moving an image on the display 102 based on tracking the eye of the user. In an example, the images on the display 102 can be moved based on the user profile 214. As noted above, the user profile 214 is based on tracking the eye of the user over a period of time to identify a favored location of focus on the display 102 or a particular window or graphical user interface 110.

As noted above, the display 102 may be an ultra-wide screen display. Thus, the user may move his or head to view different portions of the screen. The user may tend to favor a particular location or portion of the display 102 when working with the display 102.

The images 402 and 404 may be folders or icons that are displayed in the upper left-hand corner of the display 102 by default by an operating system of the computing device. However, the user profile 214 may indicate that a favored location of focus is the upper middle portion of the display 102. The display 102 may then move the images 402 and 404 to the favored location of focus based on the user profile 214. As shown in FIG. 4, the previous locations of the images 402 and 404 are illustrated in dashed lines. The present locations of the images 402 and 404 based on the user profile 214 are illustrated in solid lines.

In an example, the user may select which images or what types of images can be moved based on the user profile 214. For example, the user may select desktop folders, icons, and pop-up notifications to be moved based on the user profile 214, but prevent application windows or web browser windows from being moved based on the user profile 214.

In an example, the user profile 214 may be transmitted to a third party. The user may give permission for the third party to receive the user profile 214 or may sell the information in the user profile 214 to the third party. For example, the third party may be a search engine company or an advertisement company. The third party may offer to pay the user for the user profile 214.

The third party may receive the user profile 214 and learn where on an image 110 (e.g., also referred to as a web browser 110) the favored location of focus is for the user. A default location for an advertisement on the web browser 110 may be a top of the web browser 110. However, based on the eye-tracking information contained in the user profile 214, the third party may learn that the user tends to look more towards a bottom center of the web browser 110. For example, the user may have a tendency to read ahead quickly. Thus, the favored location of focus for the user in the web browser 110 may be the bottom center of the web browser 110. Based on the user profile 214, the third party may move an advertisement 406 from a top center of the web browser 110 to a bottom center of the web browser 110.

In an example, the image 110 may be a video. For example, the video may be a training video (e.g., also referred to as a video 110). As noted above, the HMD 106 may provide biometric data of the user. The biometric data may be analyzed to determine a cognitive load of the user. The display 102 may change the content in the video 110 based on the cognitive load of the user such that the cognitive load of the user is in a desired range.

In addition, tracking the eyes of the user may allow the display 102 to determine if the user is paying attention to the video. For example, the eye-tracking may be performed as the user is watching the video 110. During the video 110, the user may turn his or her head to talk to another person. The display may determine that the field-of-view of the user does not include the display 102 based on the images captured by the camera 104.

In response, the display 102 may pause the video 110 until the location of focus of the user is determined to be back on the video 110. In another example, an audible or visual notification may be presented to the user to have the user focus back on the video 110. In an example, the location of the video 110 may be moved to location of focus of the user based on the eye-tracking (e.g., the user may be trying to look at another window on the display 102 while the video 110 is playing). Thus, the combination of the eye-tracking and biometric data can be used for training videos to ensure that the user is paying attention and being properly trained.

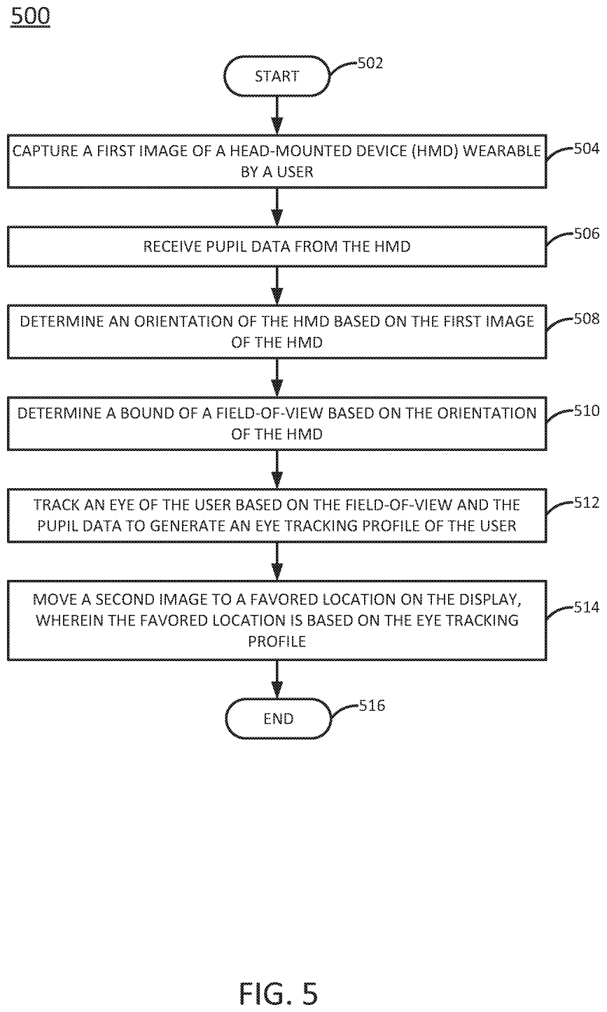

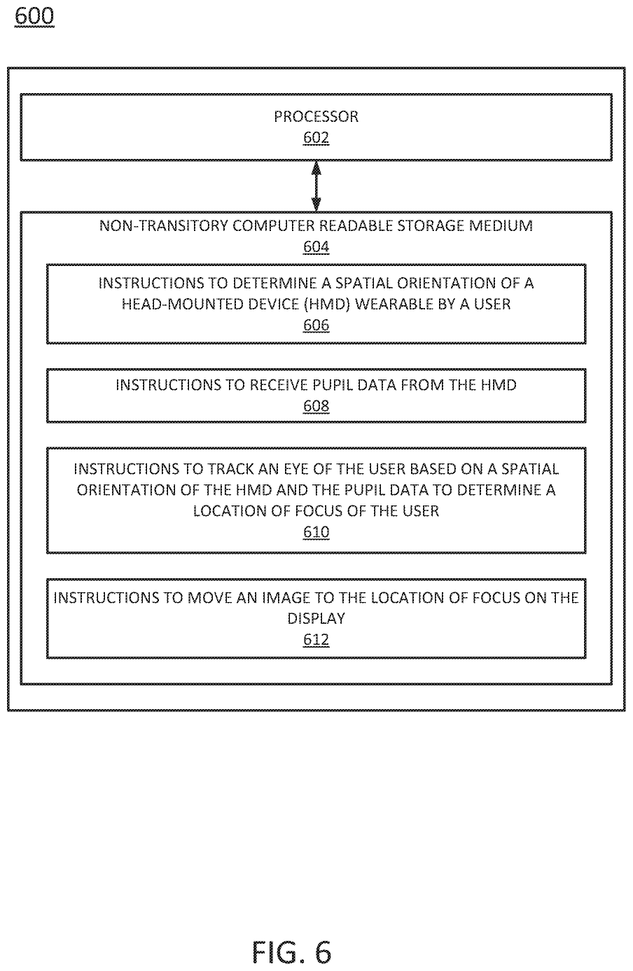

FIG. 5 illustrates a flow diagram of an example method 500 for moving an image on a display based on tracking the eye of a user of the present disclosure. In an example, the method 500 may be performed by the display 100 or the apparatus 600 illustrated in FIG. 6, and discussed below.

At block 502, the method 500 begins. At block 504, the method 500 captures a first image of a head-mounted device (HMD) wearable by a user. The image of the HMD may be captured by a camera on the display. The camera may be a red, green, blue (RGB) camera that is an external camera or built into the display. The camera may be located towards a top center of the display. The camera may be initialized such that the camera knows how far the HMD is located from the camera, learn a “centered” position where the HMD is viewing at a center of the display, and the like.

At block 506, the method 500 receives pupil data from the HMD. In an example, the pupil data may include a gaze vector. The gaze vector can be calculated by monitoring a direction that the pupils are looking. The pupil data may also include dilation information that can be analyzed to determine an emotional or cognitive state of the user.

At block 508, the method 500 determines an orientation of the HMD based on the first image of the HMD. For example, the orientation of the HMD may be left, right, up, down, or any combination thereof. The orientation of the HMD may be analyzed to determine a field-of-view of the user. For example, the centered position of the HMD may include the entire display in the field-of-view. When the orientation of the HMD is to the right the display may determine that the field-of-view includes a right portion of the display, but may not include a left portion of the display.

At block 510, the method 500 determines a bound of a field-of-view based on the orientation of the HMD. For example, based on the initialization of the camera and the orientation of the HMD in the images, the display 102 may determine the bound of the field-of-view. The bound may be an area of the field-of-view that includes a portion of the display 102. Thus, if the gaze vector is pointed at a portion in the field-of-view that is outside of the bound, the user may not be looking at anything on the display 102.

At block 512, the method 500 tracks an eye of the user based on the field-of-view and the pupil data to generate an eye-tracking profile of the user. For example, based on the field-of-view and the pupil data, the display may determine a location of focus. In an example, the location of focus may be tracked over time to create an eye-tracking profile of the user. The eye-tracking profile of the user may provide a favored location of focus of the user. For example, the favored location of focus may be a location where the user looks a number of times that is greater than a threshold number of times (e.g., the user looks at a location on the display more than 50% of the time), or may be a location where the user looks more than any other location.

At block 514, the method 500 moves a second image to a favored location on the display, wherein the favored location is based on the eye-tracking profile. In an example, the second image may be a desktop folder or icon. The second image may be moved from a default location to the favored location based on the eye-tracking profile. At block 516, the method 500 ends.

FIG. 6 illustrates an example of an apparatus 600. In an example, the apparatus 600 may be the display 102. In an example, the apparatus 600 may include a processor 602 and a non-transitory computer readable storage medium 604. The non-transitory computer readable storage medium 604 may include instructions 606, 608, 610, and 612 that, when executed by the processor 602, cause the processor 602 to perform various functions.

In an example, the instructions 606 may include instructions to determine a spatial orientation of a head-mounted device (HMD) wearable by a user. The instructions 608 may include instructions to receive pupil data from the HMD. The instructions 610 may include instructions to track an eye of the user based on a spatial orientation of the HMD and the pupil data to determine a location of focus of the user. The instructions 612 may include instructions to move an image to the location of focus on the display.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A display, comprising:
a camera to capture a first image of a head-mounted device (HMD) wearable by a user;
a communication interface to receive pupil data from the HMD; and
a processor communicatively coupled to the camera and the wireless communication interface, the processor to:
determine a bound of a field-of-view based on the first image of the HMD;
track an eye of the user based on the field-of-view and the pupil data to determine a location of focus of the user; and
move a second image to the location of focus on the display.

2. The display of claim 1, wherein the pupil data comprises a gaze vector based on eye-tracking sensors in the HMD.

3. The display of claim 1, wherein the camera comprises an external camera.

4. The display of claim 1, wherein the second image comprises a cursor.

5. The display of claim 4, wherein the processor is to track the eye of the user continuously and move the cursor in response to the location of focus as the eye is tracked.

6. The display of claim 1, wherein the second image comprises an icon and to move the second image comprises moving the icon to the location of focus.

7. The display of claim 1, wherein the second image comprises an advertisement and to move the second image comprises moving the advertisement in a web browser to the location of focus.

8. A method, comprising:
capturing a first image of a head-mounted device (HMD) wearable by a user;
receiving pupil data from the HMD;
determining an orientation of the HMD based on the first image of the HMD;
determining a bound of a field-of-view based on the orientation of the HMD;
tracking an eye of the user based on the field-of-view and the pupil data to generate an eye-tracking profile of the user; and
moving a second image to a favored location on the display, wherein the favored location is based on the eye-tracking profile.

9. The method of claim 8, wherein the tracking the eye of the user is performed for a predetermined amount of time to generate the eye-tracking profile of the user.

10. The method of claim 8, wherein the favored location is a location on the display that is focused on more than other locations on the display.

11. The method of claim 8, further comprising:
transmitting the eye-tracking profile of the user to a third party; and
receiving from the third party a graphical advertisement to display on the favored location on the display.

12. The method of claim 8, further comprising:
receiving biometric data of the user from the bio-glasses;
determining a cognitive load of the user based on the biometric data; and
adjusting the second image in the favored location on the display based on the cognitive load of the user.

13. A non-transitory computer readable storage medium encoded with instructions executable by a processor, the non-transitory computer-readable storage medium comprising:
instructions to determine a spatial orientation of a head-mounted device (HMD) wearable by a user;
instructions to receive pupil data from the HMD;
instructions to track an eye of the user based on a spatial orientation of the HMD and the pupil data to determine a location of focus of the user; and
instructions to move an image to the location of focus on the display.

14. The non-transitory computer readable storage medium of claim 13, further comprising:
instructions to compare the location of focus to a displayed image to determine the image that the user is focused on.

15. The non-transitory computer readable storage medium of claim 14, further comprising:
instructions to display a menu associated with the image that is focused on by the user.
